(19) United States
(12) Plant Patent Application Publication
(10) Pub. No.: US 2014/0223624 P1
(43) Pub. Date: Aug. 7, 2014

(54) Mint plant "Boulder Mint"

(50) ;

(71) Applicant: ESSEX LABORATORIES, INC., Napavine, WA (US)

(72) Inventor: Donald D. Roberts, Independence, OR (US)

(73) Assignee: ESSEX LABORATORIES, INC., Napavine, WA (US)

(21) Appl. No.: 13/815,160

(22) Filed: Feb. 4, 2013

(51) Int. Cl. A01H 5/00

(52) U.S. Cl. PLT/259

(57) ABSTRACT

Mint selection 07-A3-5A is a new Mentha sp. cultivar that produces an essential oil different in composition than commercially grown mint varieties. The essential oil is similar to standard mint oil in components composition but differs in the typical ratio of components. Organoleptically, it differs from typical Mentha piperita peppermint oil. It is more resistant to mint rust than current commercially grown Mentha arvensis varieties.

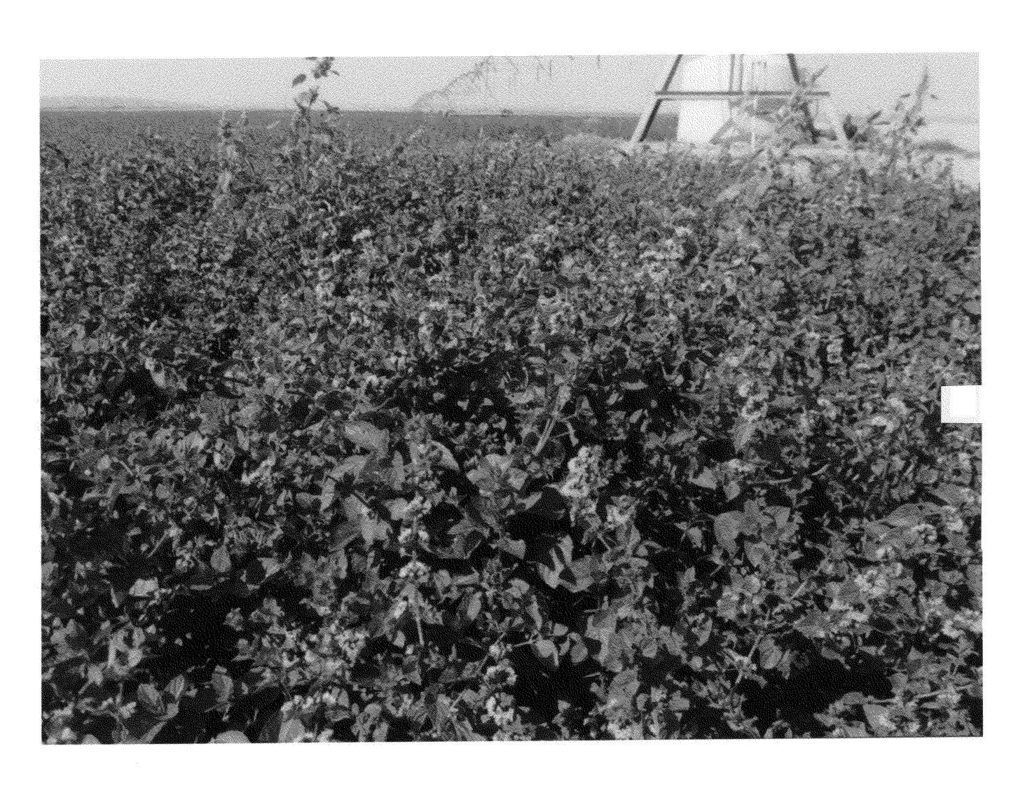

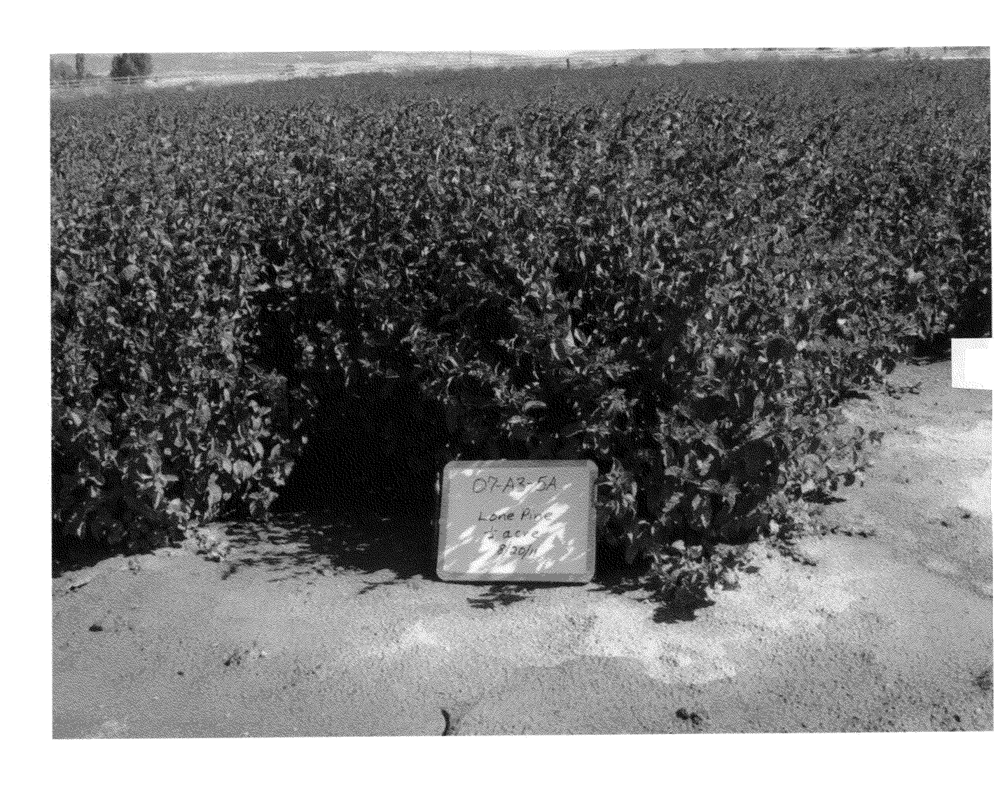

### FEDERAL SPONSORSHIP

None

### TYPE OF PLANT AND NAME OF VARIETY

The present invention relates to a new and distinct variety of peppermint plant developed from a parent of the species M. arvensis. The new variety will be identified as “Boulder Mint.”

### SUMMARY OF THE INVENTION

Mint selection 07-A3-5A denominated “Boulder Mint” is a new Mentha sp. cultivar that produces an essential oil different in composition than commercially grown mint varieties. The essential oil is similar to standard mint oil in components composition but differs in the typical ratio of components. Organoleptically, it differs from typical Mentha piperita peppermint oil. It is more resistant to mint rust than current commercially grown Mentha arvensis varieties.

### DISCOVERY AND ASEXUAL REPRODUCTION

This new mint was developed in a mint breeding program in which the primary objective was to develop a Mitcham type peppermint variety having a specific oil composition, acceptable yield and resistant to mint diseases. The new variety is as susceptible to mint wilt (soil borne fungus Verticilium dahlia) as its parent, but more resistant to mint rust (air-borne fungus Mentha puccinia). No rust has been observed on 07-A3-5A since 2007 and subsequent years. Selection 07-A3-5A has a higher yield of oil than the control variety, Black Mitcham, in test plots since 2007. This plant was selected from a population of mint seedlings in research plots on land near Monmouth, Oreg. and initially identified as 07-A3-5A.

Selection 07-A3-5A originated as a seedling from an open pollinated Mentha arvensis female parent included with other M. arvensis and male fertile M. piperita plants in a polycross breeding program. Diploid M. piperita is sterile and only becomes fertile in the polyploidy state. The parent plants in the polycross breeding system were composed of selected fertile male and female genotypes based on certain desirable characteristics.

Selection 07-A3-5A is asexually propagated to maintain the cultivars genetic integrity and as a means of increasing the selection for commercial planting. Asexual propagation, by tip cuttings or stolon sections, is a common practice in commercial mint cultivation and serves as a means of propagating the normally sterile mint plant. At the inventor's direction, Premier Botanicals has conducted asexual propagation of 07-A3-5A for greenhouse and field planting at Monmouth, Oreg., since 2007 and the genotype come true to form with each generation.

### BRIEF DESCRIPTION OF THE PHOTOGRAPHS

The accompanying color photographs show typical, field grown vegetative growth of 07-A3-5A and depicts the color as nearly as reasonably possible.

Photograph 1 illustrates the flowering pattern, multiple flowering shoots, inflorescence spike development, and leaf shape of my new mint plant in accordance with the present invention.

Photograph 2 illustrates the leaf shape and compact growth of my new mint plant.

### DESCRIPTION OF PLANT

My new mint plant improves upon and is distinct from other mint plants in several characteristics, including but not limited to, the following:

1. The ability to produce an essential oil different in composition than typical commercial M. arvensis or M. piperita, but with similar components as ‘Black Mitcham’ peppermint (M. piperita);

2. Produce more oil on a dry weight basis than currently grown mint varieties;

3. A more compact plant but with a branching pattern similar to its parent;

4. An early spring growth similar to Black Mitcham peppermint but with a more vigorous growth through maturity;

5. A level of resistance to mint rust equal to or greater than that of its M. arvensis female parent.

The essential oil extracted from 07-A3-5A has a composition of components that is different than that of commercial oil produced by Black Mitcham peppermint (M. piperita) and M. arvensis as illustrated in Table 1. The concentration of menthone in oil of 07-A3-5A is greater than that of M. arvensis and M. piperita. There is a trace of menthofuran and pulegone present in the oil of 07-A3-5A and absent in oil of its parent and commercial M. arvensis. Organoleptically, the oil of 07-A3-5A is different than that of Black Mitcham and M. arvensis, reflecting the difference in oil component ratios.

TABLE 1
A Comparison of 07-A3-5A Essential Oil collected from test
plots near Monmouth, Oregon to that of its M. arvensis parent,
Commercial M. arvensis, and Commercial M. piperita Oils.1/

| Essential Oil | Mentha arvensis | 07-A3-5A | Commercial | Commercial |
|---|---|---|---|---|
| Components | Parent seedling 2/ | Seedling2/ | M. arvensis | M. piperita |
| 1-Limonene | 3.0 | 1.6 | 3.0 | 1.7 |
| 1,8-Cineole | <1.0 | <1.0 | <1.0 | 4.9 |
| 1-Menthone | 15.7 | 44.2 | 7.3 | 19.4 |
| Menthofuran | 0.0 | <1.0 | 0.0 | 4.2 |
| Isomenthone | 3.5 | 3.1 | 3.6 | 3.1 |
| 1-Menthyl | 3.7 | 3.5 | 2.9 | 5.3 |
| Acetate | | | | |
| 1-Menthol | 64.3 | 34.1 | 73.9 | 44.6 |
| Pulegone | <1.0 | 1.9 | 0.0 | 2.1 |

The numbers listed in the above table are percentages based upon the analysis of the respective mint oils by gas chromatography. The percentages are determined by calculation of the relative peak areas.
1/Commercial essential oils of M.arvensis and M.piperita were what is typically produced by mint growers.
2/The essential oil of M. arvensis parent seedling and 07-A3-5A were collected from plants growing in test plots in 2007.

Taxonomic Description of 07-A3-5A

This new plant, under greenhouse and field growing conditions, is a bush type plant with lateral branches at each node of the main stems. The height of 07-A3-5A is equal to or greater than Black Mitcham growing under similar conditions and will vary based on fertilizer, soil quality, and water application, amongst other known factors that affect growth patterns. Secondary and tertiary branching occurs to form a compact growth habit. The main stem at mid-plant (approximately between the eleventh and twelfth node) of a mature plant is 4.4-5.2 mm in width. The secondary and tertiary branch stems are 1.9-2.2 mm and 1-2 mm in width, respectively.

Mature leaves at the bottom of the plant are ovate lanceolate as are leaves on secondary branch stems. Leaves on upper mature plants, both main and secondary stems are more lanceolate (Photograph 3). Mid-main stem leaf size at flowering is 30-34 mm in width and 63-68 mm in length. Leaf size on secondary branches at flowering is 18-24 mm in width and 38-44 mm in length. Leaf petioles on the main stem leaves are 10-16 mm in length while petioles on secondary branch stem leaves are 4-7 mm in length. Leaves on the mid-main and lower stem tend to be less dentate than Black Mitcham peppermint leaves while the leaves on the upper plant tend to be more dentate and similar to Black Mitcham peppermint leaves. The main stem leaves have from 16 to 18 teeth on each side while the secondary branch leaves have 10-14 teeth on each side. The leaf is green in color, ranging from Royal Horticulture Society Color Code N 137B to N137C in the green group classification. The leaf has 6-9 lateral veins, more or less in parallel off the main vein that runs from the petiole to the tip of the leaf. The veins are prominent in all leaves of 07-A3-5A.

The inflorescence is a conspicuous spike with capitate flowers developing at the nodes of the spike stem. The cylindrical spikes are about 30 mm in diameter and is indeterminate. The capitate flowers are 15-20 mm in width and 10-15 mm in length. The flowers consist of five petals fused into a two lipped corolla. The corolla is white in color as illustrated in The Royal Horticulture Society Color Code 84D in the Violet Group The calyx is generally yellow green and is 143A to 143B RHS, Green Group as illustrated in The Royal Horticulture Color Code index. The gynoecium consists of a single pistil with two lobed stigma that is exserted. The androecium consists of four stamens, each with a distinct filament and anther.

While the plant that comprises the present invention has been described in connection with a specific embodiment thereof, it will be understood that this application is intended to cover any variation, uses, or adaptation of the invention (particular those induced by cultivation under different environmental conditions) following, in general the principles of the invention and including such departures from the present disclosure as come within known or customary practice in the art to which the invention pertains and as may be applied to the essential features hereinbefore set forth, and as fall within the scope of the invention and the limits of the appended claim.

I claim:

1. A new and distinct variety of peppermint plant, substantially as shown and described, characterized particularly by improving resistance to mint rust, and producing a unique essential oil.
